(19) United States
(12) Patent Application Publication
(10) Pub. No.: US 2012/0232531 A1
(43) Pub. Date: Sep. 13, 2012

(54) PROCESS FOR PRODUCING PLASTIC RODS

(75) Inventors: Rainer Goering, Borken (DE); Markus Hartmann, Sendenhorst (DE); Karl Kuhmann, Duelmen (DE); Bernfried Kalthof, Kleinwallstadt (DE)

(73) Assignee: Evonik Degussa GmbH, Essen (DE)

(21) Appl. No.: 13/396,643

(22) Filed: Feb. 15, 2012

(30) Foreign Application Priority Data
Feb. 17, 2011 (DE) 102011004305.5

(51) Int. Cl. A61B 17/00; B32B 3/26; B29C 47/06

(52) U.S. Cl. 606/1; 264/172.15; 264/45.9; 428/364

(57) ABSTRACT

A process for producing a plastic rod, in particular round rods, suitable for machining is provided. The process includes extruding a plastic profile of a first plastic molding composition which forms the outermost layer of the plastic rod, and inserting a second plastic molding composition as a rod core into the profile within a calibrator downstream of the extruder. The first plastic molding composition contains at least 50% by weight of a semicrystalline thermoplastic and the first plastics moulding composition has the following properties: a) crystallite melting point Tₘ in accordance with ISO 11357 of at least 170° C., b) crystallization temperature Tₖ in accordance with ISO 11357 of at most 70 K below T. and c) enthalpy of fusion AH in accordance with ISO 11357 of at least 20 J/g.

### CROSS REFERENCE TO RELATED APPLICATIONS

This application claims priority to German Application No. 102011004305.5, filed Feb. 17, 2011, the disclosure of which is incorporated herein by reference in its entirety.

### BACKGROUND OF THE INVENTION

The present invention relates to a process for extruding rods made of semicrystalline thermoplastics. The rods may be compact, which is termed solid rods. In a special case, the present invention relates to round rods which in accordance with EN ISO 15860, are extruded, cast or compression-moulded elongate, straight, non-hollow products with a circular cross section that remains the same over their entire length.

Rods made of thermoplastics are preferably used for producing finished parts by machining, in particular finished parts with relatively small numbers of units, prototype parts or parts which the injection-moulding process or other plastics-processing methods can produce only with difficulty or are almost incapable of producing.

From the point of view of the user, the manufacture of components of this type from high-melting-point thermoplastics, such as polyether ether ketone (PEEK) is rather similar to traditional metalworking: standardized semifinished products, such as tubes, profiles or rods are machined to size to give the desired shape. Traditional methods of plastics manufacture are used only to produce the semifinished products: profiles, tubes or solid rods are therefore in principle extruded in an extrusion plant in just the same way as is conventional during the production of corresponding semifinished products made of polypropylene (PP) or polyethylene (PE).

Plastic rods with relatively small diameters can be obtained with good quality through conventional extrusion of a thermoplastic moulding composition. However, this process becomes difficult for rods starting at a diameter of about 20 mm, depending on the moulding composition used, since a large amount of thermal energy has to be dissipated from the extruded rod by cooling, and adequate dimensional stability can therefore be achieved only after a long cooling time, and this has the disadvantageous effect that the rod's own weight can cause it to sag with a resultant change of cross section, or deflection can occur. If the extrusion speed is reduced in order to create better cooling conditions for the rod, manufacturing costs are increased to values that become economically unacceptable.

DE 10 2004 015 072 A1 describes division of a plastics melt into two streams in the die during the extrusion process, and the externally arranged melt here is used in a conventional method to extrude a tube. The second melt is inserted by way of a lance into the tube. To prevent collapse of the tube, support air is introduced by way of the head and subatmospheric pressure is applied at the calibration system. It is indicated that this process can manufacture round bars which have excellent optical properties and particularly high dimensional accuracy in cross section.

DE 37 18 036 A1 describes a process for producing mouldings with large cross-section dimensions, for example shock absorbers or protective coverings made of polyethylene, by multilayer extrusion. A first stream of molten material is used here to mould a hollow profile, which is cooled in a calibrator jacket, while a second stream of molten material is extruded into the cavity. The melt here is injected approximately at the position where the profile leaves the calibration system. However, the exterior hollow profile which has solidified but which still has a degree of flexibility expands to a convex shape during this process, thus accepting a somewhat greater volume of material than would actually be desirable. The subsequent shrinkage during cooling inhibits the formation of cavities or vacuoles.

Conventionally known methods as described, are however, unsatisfactory. DE 10 2004 015 072 A1 relates exclusively to amorphous thermoplastics. If an attempt is made to process semicrystalline thermoplastics in this manner, the relatively high shrinkage of these materials is in danger of causing cavities to form during cooling, these being unacceptable for further processing. The approach of DE 37 18 036 Al, inhibiting the formation of cavities by using controlled expansion, becomes useless when exact dimensional accuracy is important.

### SUMMARY OF THE INVENTION

The object of the present invention consists in producing plastic rods made of semicrystalline thermoplastics which comply with the following requirements:
an improvement over conventional extrusion should be achieved in respect of dimensional accuracy of diameter for rods starting from a diameter of about 20 mm. In the ideal case, the values should very substantially achieve or be within the tolerances prescribed by the standards DIN 16980 and EN ISO 15860;
the deviation of the rod from a straight axial line, based on its external edges and one meter of length, should likewise be minimized. Deviations from straightness can cause problems during manufacture by machining;
a solid rod should have no cavities or vacuoles, since these would be highly problematic during further processing and for the function of the subsequent finished part;
a solid rod should moreover have the lowest possible level of internal stresses, in order to facilitate machining and to reduce the warpage that can arise through mechanical processes. The level of internal stresses can be assessed from the warpage of thin sections carefully cut from the solid rods;
finally, it should be possible to produce multilayer rods in which the layers are composed of different materials.

This and other objects have been achieved by the present invention, the first embodiment of which includes A process for producing a plastic rod having a shape, the

plastic rod comprising:

at least an outermost profile layer; and

an inner core rod within the profile;

the process comprising:

supplying at least a first plastic molding composition and at least a second plastic molding composition to an extruder having at least an outermost profile die and a inner rod die;

extruding the at least first plastic molding composition through the profile die to form a profile outermost layer of the rod;

passing the extruded profile to a calibrator;

extruding the second plastic molding composition through a rod die to form a core rod;

inserting the extruded core rod into the profile within the calibrator to form a newly formed rod;

calibrating, drawing off and cooling the newly formed rod to a shape to obtain the plastic rod;

wherein

the second plastic molding composition comprises a thermoplastic;

the first plastic molding composition comprises at least 50% by weight of a semicrystalline thermoplastic, and

a crystallite melting point Tₘ of the first plastic molding composition is at least 170° C. in accordance with ISO 11357,
a crystallization temperature Tₖ of the first plastic molding composition is at most 70 K below Tₘ in accordance with ISO 11357, and

an enthalpy of fusion ΔH of the first plastic molding composition is at least 20 J/g in accordance with ISO 11357.

In a second embodiment, the shape of the plastic rod is round.

In a further embodiment, the present invention includes a medical implant obtained by machining the plastic rod according to the present invention and in further embodiments the present invention includes medical implants having foamed portions.

### DETAILED DESCRIPTION OF THE PREFERRED EMBODIMENTS

In a first embodiment, the present invention provides a process for producing a plastic rod having a shape, the plastic rod comprising:

at least an outermost profile layer; and

an inner core rod within the profile;

the process comprising:

supplying at least a first plastic molding composition and at least a second plastic molding composition to an extruder having at least an outermost profile die and a inner rod die;

extruding the at least first plastic molding composition through the profile die to form a profile outermost layer of the rod;

passing the extruded profile to a calibrator;

extruding the second plastic molding composition through a rod die to form a core rod;

inserting the extruded core rod into the profile within the calibrator to form a newly formed rod;

calibrating, drawing off and cooling the newly formed rod to a shape to obtain the plastic rod; wherein the second plastic molding composition comprises a thermoplastic; the first plastic molding composition comprises at least 50% by weight of a semicrystalline thermoplastic, and a crystallite melting point Tₘ of the first plastic molding composition is at least 170° C. in accordance with ISO 11357, a crystallization temperature Tₖ of the first plastic molding composition is at most 70 K below Tₘ in accordance with ISO 11357, and an enthalpy of fusion AH of the first plastic molding composition is at least 20 J/g in accordance with ISO 11357.

The crystallite melting point Tₘ of the first plastic molding composition may be at least 170° C., preferably at least 185° C., particularly preferably at least 200° C. and with particular preference at least 215° C.

The crystallization temperature Tₖ of the first plastic molding composition may be at most 70 K below Tₘ, preferably at most 60 K below Tₘ, particularly preferably at most 55 K below Tₘ and with particular preference at most 50 K below Tₘ.

The enthalpy of fusion ΔH of the first plastic molding composition may be at least 20 J/g, preferably at least 25 J/g, particularly preferably at least 30 J/g, with particular preference at least 35 J/g and very particularly preferably at least 40 J/g, as measure of the degree of crystallization.

Tₘ, Tₖ and ΔH may be determined in accordance with ISO 11357 by heating from room temperature to at most 390° C. at a heating rate of 20 K/min, cooling to −60° C. at a heating rate of 20 K/min and heating again to at most 390° C. at a heating rate of 20 K/min. Tₘ and ΔH are determined during the 2^{nd} heating procedure, whereas Tₖ is determined during the cooling procedure. The maximum heating temperature depends on thermal stability, and also on Tₘ; it may be advisable here to exceed Tₘ by at least about 60 K.

The crystallite melting point Tₘ of the first plastics moulding composition must be at least 170° C., in order to ensure sufficiently rapid solidification of the extruded profile. However, this condition may not be sufficient in itself. If crystallization were delayed, the flexibility of the profile after leaving the calibration system may still be sufficient to cause expansion resulting from the melt pressure of the filler material. For this reason it is also necessary to maximize rapidity of onset of crystallization during cooling, i.e. to maximize Tₖ. The degree of crystallization must moreover be sufficiently high to ensure the desired solidification. This may be correlated experimentally with the enthalpy of fusion.

The plastic rods produced according to the invention may be round rods; however, other geometries are also possible, examples of cross sections being oval, elliptical or polygonal (e.g. triangular, square, rectangular, rhombic, trapezoidal, pentagonal or hexagonal). In the case of a round rod, the profile extruded is a tube. The profile may have one layer and therefore be composed entirely of the first plastics molding composition, or have a plurality of layers, where at least the outermost layer is composed of the first plastics molding composition.

The first plastics moulding composition is based on a semicrystalline thermoplastic which by way of example may be a polyamide, a semiaromatic polyester, a fluoropolymer such as perfluoroalkoxy (PFA), ethylene tetrafluoroethylene (ETFE), ethylene fluoroethylene propylene (EFEP), polyvinylidene fluoride (PVDF), polytetrafluoroethylene (PTFE), a polyphenylene sulphide or a polyarylene ether ketone. A factor requiring consideration during the selection of the thermoplastic may be that in particular the crystallization temperature Tₖ and the enthalpy of fusion are affected by the other constituents of the moulding composition. Tₖ may by way of example be raised significantly by adding nucleating agents.

Polyamide may be produced from a combination of diamine and dicarboxylic acid, from an ω-aminocarboxylic acid or from the corresponding lactam. In principle, it is possible to use any sufficiently semicrystalline polyamide, such as PA6, PA66 or copolyamides using this basis with units deriving from terephthalic acid and/or from isophthalic acid (the general term used being PPA), or else PA9T and PA10T and blends of these with other polyamides. Examples of other suitable polyamides are PA610, PA88, PA8, PA612, PA810, PA108, PA9, PA613, PA614, PA812, PA128, PA1010, PA10, PA814, PA148, PA1012, PA11, PA1014, PA1212 and PA12. It is also possible, of course, to use copolyamides based thereon. Methods to produce polyamides are conventionally known.

It may be equally possible to use mixtures of various polyamides, with the proviso that compatibility is sufficient. Compatible polyamide combinations are known to the person skilled in the art; and examples that may be mentioned here are the combinations PA12/PA1012, PA12/PA1212, PA612/PA12, PA613/PA12, PA1014/PA12 and PA610/PA12, and also corresponding combinations with PA11. Compatible combinations may be determined by routine experimentation.

Thermoplastic polyesters may be produced by polycondensation of diols with dicarboxylic acids or with polyester-forming derivatives thereof, examples being dimethyl esters. Suitable diols have the formula HO—R—OH, where R is a divalent, branched or unbranched aliphatic and/or cycloaliphatic moiety having from 2 to 18, preferably from 2 to 12, carbon atoms. Suitable dicarboxylic acids have the formula HOOC—R′—COOH, where R′ is a divalent aromatic moiety having from 6 to 20, preferably from 6 to 12, carbon atoms.

Examples of diols are ethylene glycol, trimethylene glycol, tetramethylene glycol, 2-butene-1,4-diol, hexamethylene glycol, neopentyl glycol, and also cyclohexanedimethanol. The diols can be used alone or as diol mixture.

Examples of aromatic dicarboxylic acids include terephthalic acid, isophthalic acid, 1,4-, 1,5-, 2,6- or 2,7-naphthalenedicarboxylic acid, biphenyl-4,4′-dicarboxylic acid and diphenyl ether 4,4′-dicarboxylic acid. Up to 30 mol % of these dicarboxylic acids may be replaced by aliphatic or cycloaliphatic dicarboxylic acids having from 3 to 50 carbon atoms and preferably having from 6 to 40 carbon atoms, e.g. succinic acid, adipic acid, sebacic acid, dodecanedioic acid or cyclohexane-1,4-dicarboxylic acid.

Examples of suitable polyesters include polypropylene terephthalate, polybutylene terephthalate, polyethylene 2,6-naphthalate, polypropylene 2,6-naphthalate and polybutylene 2,6-naphthalate.

Methods to produce these polyesters are conventionally known (DE-A 24 07 155, 24 07 156; Ullmann's Enzyklopadie der technischen Chemie [Ullmann's Encyclopaedia of Industrial Chemistry], 4th Edition, Volume 19, pp. 65 ff., Verlag Chemie, Weinheim, 1980).

The fluoropolymer may by way of example be a polyvinylidene fluoride (PVDF), an ethylene-tetrafluoroethylene copolymer (ETFE), an ETFE modified by a tercomponent, for example propene, hexafluoropropene, vinyl fluoride or vinylidene fluoride (an example being EFEP) or a tetrafluoroethylene-perfluorinated alkyl vinyl ether copolymer (PFA).

Polyphenylene sulphide comprises units of the formula

(—C₆H₄—S—);

Preferably, the polyphenylene sulphide comprises at least 50% by weight, at least 70% by weight, or at least 90% by weight, of these units. The remaining units may be those given below for the polyarylene ether ketone, or tri- or tetrafunctional branching agent units which result from the concomitant use of, for example, trichlorobenzene or tetrachlorobenzene during the synthesis.

Polyphenylene sulphide is available commercially in a wide variety of types or molding compositions.

Polyarylene ether ketone comprises units of the formulae

(—Ar—X—) and (—Ar′—Y),

where Ar and Ar′ are a divalent aromatic moiety, preferably 1,4-phenylene, 4,4′-biphenylene, or else 1,4-, 1,5- or 2,6-naphthylene. X is an electron-withdrawing group, preferably carbonyl or sulphonyl, while Y is another group, such as O, S, CH₂, isopropylidene or the like. At least 50%, preferably at least 70% and particularly preferably at least 80%, of the groups X here are a carbonyl group, while at least 50%, preferably at least 70% and particularly preferably at least 80% of the groups Y are composed of oxygen.

In a preferred embodiment, 100% of the groups X are carbonyl groups and 100% of the groups Y are oxygen. The polyarylene ether ketone may by way of example be a polyether ether ketone (PEEK; formula I), a polyether ketone (PEK; formula II), a polyether ketone ketone (PEKK; formula III) or a polyether ether ketone ketone (PEEKK; formula IV), but other arrangements of the carbonyl groups and oxygen groups may also be possible.

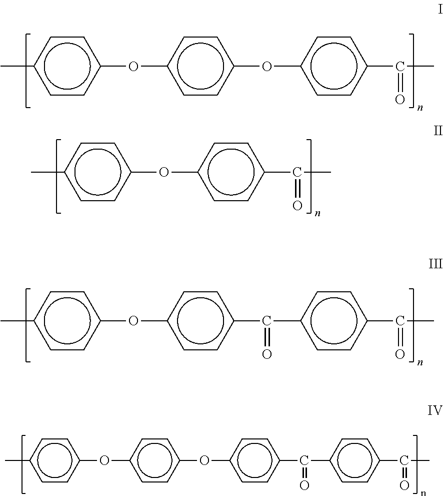

The polyarylene ether ketone is semicrystalline, and this is discernible by way of example in DSC analysis through appearance of a crystallite melting point Tₘ, which in most instances may be about of the order of magnitude of 300° C. or greater.

The molding compositions may each independently comprise further components, e.g. impact modifiers, other thermoplastics, plasticizers and other conventional additives, for example pigments or fillers, such as carbon black, titanium dioxide, zinc sulphide, reinforcing fibres, e.g. fibres of glass or of carbon, or whiskers, lubricants, such as graphite, molybdenum disulphide, boron nitride or PTFE, nucleating agents, such as talc powder, processing aids, such as waxes, zinc stearate or calcium stearate, antioxidants, UV stabilizers, and also additions which give the product antielectrostatic properties, e.g. carbon fibres, graphite fibrils, stainless-steel fibres, or conductive carbon black.

In one embodiment of the present, the first and second molding compositions are identical and in this embodiment, a melt stream may advantageously be divided, as described in DE 10 2004 015 072 A1.

In another embodiment, the first and second molding compositions are not identical, but the same definition according to the claims covers the second moulding composition and the first moulding composition. This is in particular the case when the underlying thermoplastic composition is identical in the two cases, but the molding compositions differ in type and/or amount of the other constituents. In other cases the two thermoplastics may be different but mutually compatible.

In a further embodiment, the second composition may not be covered by the same definition according to the claims which covers the first moulding composition. It may be semicrystalline or amorphous. Examples that may be mentioned are polypropylene, polyethylene terephthalate, polycarbonate, fully aromatic polyesters, polyester carbonate, polysulfone, polyether sulfone, polyphenyl sulfone, polyphenylene ether, polyetherimide, polyimide and transparent polyamides, other examples being blends made of polyarylene ether ketone with optionally relatively large proportions of polysulfone, polyether sulfone, polyphenyl sulfone, polyetherimide and/or polyimide. The second moulding composition may comprise the conventional additives as previously listed for the first molding composition. With a view to good coupling of the two materials, there must be adequate adhesion compatibility between the two molding compositions.

In one preferred embodiment, the profile may be foamed. The foaming may take place chemically through addition of a blowing agent susceptible to decomposition, or may take place physically by virtue of a blowing gas metered into the material. The degree of expansion, defined by the ratio of the density of the foam to the density of the unfoamed molding composition, may preferably be from 1.01 to 10. The foam may have open cells or closed cells. Various combinations of the embodiments described herein may be envisioned by one of ordinary skill in the art and all such combinations are within the embodiments according to the present invention.

In a further preferred embodiment, the second plastics molding composition may foamed to form open or closed cells. Again, the various combinations of the embodiments described herein which may be envisioned by one of ordinary skill in the art are within the embodiments according to the present invention.

Depending upon the use for which the plastic rod is intended, it may be required that the profile and the core rod adhere securely to one another. To this end, the two molding compositions must have sufficient mutual compatibility. In cases where compatibility is not sufficient, the profile may be coextruded in a plurality of layers, where the innermost layer may be composed of a suitable adhesion promoter. In the case of PA12 (profile) and PA6 (core rod), compatibility may for example be improved through an adhesion promoter layer made of PA612, while in the case of PA612 (profile) and polyethylene terephthalate (PET; core rod) compatibility may be obtained, for example, by an adhesion promoter layer made of a PA612/PET blend which comprises PA612/PET block copolymers.

In order to achieve good adhesion, the temperature level of the core rod-material melt inserted should moreover be at least in the region of Tₘ of the material that forms the inner surface of the extruded profile.

A calibrator placed downstream of the extruder may serve to shape the external layer. It may be possible, for example, to employ a typical brass calibration system conventionally used in pipe extrusion. The calibration system may optionally either have intensive cooling to provide rapid dissipation of heat or have heating in order to provide relatively slow cooling and thus reduce the stresses arising in the plastic rod. Temperature control of the calibrator may provide for adjustment of the temperature of the extruded plastics profile so as to ensure secure adhesion of the second molding composition to the first. Such approach may be especially effective if the profile has thin walls.

The second plastics moulding composition may be introduced as melt at a location which is preferably within about 1% to about 99% of the length of the calibrator, particularly preferably within about 10% to about 90%, with particular preference within about 20% to about 80% and very particularly preferably within about 30% to about 70%. In special cases, and specifically when the extruded profile has comparatively thick walls and/or the first plastics moulding composition has a comparatively high modulus of elasticity and therefore high stiffness, it may also be possible to delay introduction of the second molding composition until after the calibrator, without any expansion of the profile taking place. The melt pressure of the second plastics moulding composition may be from 2 to 8 bar; only very slight variations in this are permitted, in order that vacuole-free rods are reliably produced. The pressure may be less for production of foamed cores.

Downstream of the calibrator there may be a cooling and/or conditioning section arranged with various cooling media or heating media. A water-immersion bath may be used here, as also can spray baths and/or air from a blower. Rods of relatively large diameter, or solid rods made of fibre-filled moulding compositions, are in particular conditioned or carefully cooled, in order to avoid excessive internal stresses and to avoid resultant premature failure of the rod.

The take-off speed generally depends on the diameter and on the molding composition. Smaller diameters may be produced at higher speeds.

The process according to the invention may be combined with a flexible calibration system, thus providing a stepless method of producing different rod diameters. Calibration systems of this type are conventionally used in the extrusion of tubes (e.g. WO 2004/091891, DE 10 2004 029 498 B3, EP 1 627 724 A2, WO 00/16963 and WO 2005/018910). Such use of a flexible calibration system may of economic interest since the manufacture of components includes machining processes to bring the plastic rod to the desired dimension. In particular for high-value rods made of expensive moulding compositions, there is economic value in minimizing the amount that has to be machined from the external diameter. The effect of this, where solid rods are used commercially, is that by way of example in the diameter range from about 5 mm to about 40 mm diameters are available in 1 mm steps, and production batch sizes are frequently very small.

In the context of the invention it may be possible to produce rods with a diameter (or a smallest diameter in cases where a round cross section is not involved) of from 15 to 500 mm, preferably from 20 to 400 mm and particularly preferably from 25 to 350 mm. The wall thickness of the profile may usually be from 0.2 to 25% of the diameter, preferably from 0.4 to 20% of the diameter, particularly preferably from 0.6 to 16% of the diameter and with particular preference from 0.8 to 14% of the diameter. If there is no round cross section involved, “diameter” means the smallest diameter.

In the context of the invention, multilayer rods, and in particular multilayer round rods, may be of particular interest. Examples of such embodiments may include the following:
A wear-resistant or anti-abrasive external profile layer and a tough core rod may be excellent preconditions for uses such as gear wheels, shafts or axles, an example being an external PEEK moulding composition with 30% of carbon fibres or with respectively 10% of carbon fibres, graphite and PTFE or with 30% of ceramic filler and an internal unreinforced PEEK moulding composition.
High strength of material and smooth surfaces may be combined in a solid core rod with a fibre-filled core material and an unfilled external profile layer.
In another embodiment, an external profile layer with good machinability is selected, for example made of a material which is the same as that of the core rod, but with lower molecular weight.
In order to reduce the cost of materials, a solid plastic rod may be composed of a high-value external profile layer (for example PEEK) and of a less expensive core rod component, for example a PEEK blend with PFA, polyimide, polyetherimide, LCP, for example liquid-crystalline polyester, polysulfone, polyether sulfone, polyphenyl sulfone, polybenzimidazole (PBI) and/or other polymers resistant to high temperatures.
In another embodiment, a rod may be produced from a hard compact external profile layer and a foamed core rod. In such process a foaming material is inserted into the space available in the tube; the degree of expansion and the nature of the cells may be controlled in a known manner by using the selected parameters, such as melt stiffness, amount of blowing agent, type of blowing agent, etc. This method may yield a uniform cell diameter across the entire cross section. A primary objective here is weight reduction, for example for lightweight construction uses in aerospace.
Another embodiment of particular interest for medical applications, such as, for example, for implants, may be a foamed external profile layer with a hard or tough core rod. Foamed external layers may improve angiogenesis or osseointegration, while the strong, stable core rod provides load-bearing function. Open-cell foams may be advantageous here. One possible embodiment for medical application may be a part produced from a plastic rod according to the present invention having a compact core rod made of an unfilled PEEK molding composition and an external profile layer of an open-cell foamed PEEK moulding composition with a number-average cell diameter of from 1 μm to 700 μm, preferably from 10 μm to 500 μm.
It a further preferred embodiment the present invention includes foaming both the profile and the core rod. Optionally the foam structure of the profile and core rod may be the same or different. In the case of implants, when a different level of osseointegration is required or can be achieved, it is thus possible to produce an implant with different cell structures, for example made of a PEEK molding composition, in order to bring about controlled integration.

In a highly preferred embodiment the present invention includes a method to produce a medical implant comprising the method to produce a plastic rod according to the present invention wherein at least one of the first and second molding compositions comprises PEEK.

Adequate dimensional stability may be achieved more rapidly in the extruded profile with the process according to the invention than according to conventionally known methods. The insertion of the second moulding composition as a core rod may therefore take place at a distance from the end of the calibration system such that a sufficiently long hardening time is available to the core material under melt pressure, without any expansion of the profile. This may effectively inhibit the formation of vacuoles and thus the dimensional accuracy of the plastic rod according to the present invention may be improved over rods conventionally produced.

Having generally described this invention, a further understanding can be obtained by reference to certain specific examples which are provided herein for purposes of illustration only, and are not intended to be limiting unless otherwise specified.

### EXAMPLES

The following moulding compositions were used in the inventive examples and comparative examples:

PA12: VESTAMID® LX9030 yellow with the following properties: Tₘ=176° C., Tₖ=149° C. and ΔH=64 J/g, in each case determined by the DCS method in accordance with ISO 11357;

PEEK: VESTAKEEP® 4000G of with the following properties: Tₘ=336° C., Tₖ=281° C. and ΔH=42 J/g, in each case determined by the DSC method in accordance with ISO 11357.

### Comparative Example 1

Extrusion of a solid rod of diameter 65 mm from PA 12 by standard extrusion was performed. Table 1 gives the experimental conditions used and the result obtained.

### Inventive Example 1

Extrusion of a solid rod of diameter 65 mm from PA 12 by using a two-layer die; external layer thickness 5 mm; with the lance terminating at 70% of calibrator length, was performed. See Table 1 for the experimental conditions used and the result obtained.

### Comparative Example 2

Extrusion of a solid rod of diameter 65 mm from PEEK by standard extrusion was performed; see Table 2 for the experimental conditions used and the result obtained.

### Inventive Example 2

Extrusion of a solid rod of diameter 65 mm from PEEK by using a two-layer die; external layer thickness 5 mm; with the lance terminating at 70% of calibrator length, was performed. See Table 2 for the experimental conditions used and the result obtained.

TABLE 1
Experiments using PA 12

| | Comparative Example 1 | Inventive Example 1 |
|---|---|---|
| Temperature profile in | 200° C., 220° C., 240° C., 240° C., | The two extruders had the same |
| barrel and die | 240° C. | temperature settings, namely 200° C., |
| | | 220° C., 240° C., 240° C., 240° C. |
| Screw rotation rate | 35 rpm (higher rotation rates led to | Extruder 1 for external layer: 70 rpm |
| | pressure variations, extending as far | Extruder 2 for core: 120 rpm |
| | as blocking of the solid rod in the | |
| | calibration system) | |
| Take-off speed | 0.5 m/h | 0.6 m/h |
| Melt pressure | 2-4 bar. Higher pressures led to | Extruder 1 (external layer) |
| | problems (see under screw rotation rate) | significantly greater than 4 bar |
| | | Extruder 2 about 5 bar for |
| | | inserting melt into the tube |
| Vacuum | None | 0.1-0.3 bar (for fixing the external |
| | | layer, to avoid tube collapse) |
| Diameter of solid rod: | 2.0 mm | 0.4 mm |
| cross-sectional deviations | | |
| from roundness | | |

TABLE 2
Experiments using PEEK

| | Comparative Example 2 | Inventive Example 2 |
|---|---|---|
| Temperature profile in | 320° C., 340° C., 350° C., 360° C., | The two extruders had the same |
| barrel and die | 360° C. | temperature settings, namely 320° C., |
| | | 340° C., 350° C., 360° C., 360° C. |
| Screw rotation rate | 70 rpm | Extruder 1 for external layer: 70 rpm |
| | | Extruder 2 for core: 60 rpm |
| Take-off speed | 1.68 m/h | 3.3 m/h |
| Melt pressure | 2-4 bar. Pressures above 6 bar without | Extruder 1 significantly greater |
| | subsequent conditioning led to failure of | than 4 bar |
| | the solid rod caused by internal stresses | Extruder 2 about 5 bar |
| Vacuum | None | 0.2-0.48 bar |
| Diameter of solid rod: | 1.8 mm | 0.4 mm |
| cross-sectional deviations | | |
| from roundness | | |

1. A process for producing a plastic rod having a shape, the plastic rod comprising:
at least an outermost profile layer; and
an inner core rod within the profile;
the process comprising:
supplying at least a first plastic molding composition and at least a second plastic molding composition to an extruder having at least an outermost profile die and a inner rod die;
extruding the at least first plastic molding composition through the profile die to form a profile outermost layer of the rod;
passing the extruded profile to a calibrator;
extruding the second plastic molding composition through a rod die to form a core rod;
inserting the extruded core rod into the profile within the calibrator to form a newly formed rod;
calibrating, drawing off and cooling the newly formed rod to a shape to obtain the plastic rod;
wherein
the second plastic molding composition comprises a thermoplastic;
the first plastic molding composition comprises at least 50% by weight of a semicrystalline thermoplastic, and
a crystallite melting point Tₘ of the first plastic molding composition is at least 170° C. in accordance with ISO 11357,
a crystallization temperature Tₖ of the first plastic molding composition is at most 70 K below Tₘ in accordance with ISO 11357, and
an enthalpy of fusion AH of the first plastic molding composition is at least 20 J/g in accordance with ISO 11357.

2. The process according to claim 1, wherein the shape of the plastic rod is round.

3. The process according to claim 1, wherein the semicrystalline thermoplastic of the first plastic molding composition is at least one selected from the group consisting of a polyamide, a semiaromatic polyester, a fluoropolymer, a polyphenylene sulphide and a polyarylene ether ketone.

4. The process according to claim 1, wherein the thermoplastic of the second plastic molding composition is at least one selected from the group consisting of a polyamide, a semiaromatic polyester, a fluoropolymer, a polyphenylene sulphide, a polyarylene ether ketone, a transparent polyamide, a polypropylene, a polyethylene terephthalate, a polycarbonate, an aromatic polyester, a polyester carbonate, a polysulfone, a polyether sulfone, a polyphenyl sulfone, a polyphenylene ether, a polyether imide, and a polyimide.

5. The process according to claim 4, wherein the second plastic molding composition is a blend of a polyarylene ether ketone with at least one of a polysulfone, a polyether sulfone, a polyphenyl sulfone, a polyetherimide and a polyimide.

6. The process according to claim 1, wherein the second plastic molding composition is identical to the first plastic molding composition.

7. The process according to claim 1, further comprising:
foaming the first plastic composition to form a foamed profile.

8. The process according to claim 1, further comprising:
foaming the second plastic composition to form a foamed core rod.

9. The process according to claim 1, wherein the profile die comprises a plurality of dies, and the profile comprises:
a plurality of layers, wherein the innermost layer comprises an adhesion promoter.

10. The process according to claim 1, wherein a smallest diameter of the plastic rod is from 15 to 500 mm.

11. The process according to claim 1, wherein a thickness of the plastic profile is from 0.4 to 20% of a smallest diameter of the plastic rod.

12. A method for producing a machined plastic part, comprising:
machining a plastic rod obtained according to the process of claim 1.

13. A medical implant obtained according to the method of claim 12, wherein the core rod comprises at least one polyether ether ketone molding composition.

14. The medical implant according to claim 13, wherein at least one of the profile and the core rod is foamed.

15. The medical implant according to claim 14, comprising:
an unfoamed core rod; and
an open-cell foamed profile;
wherein a number-average cell diameter of the open-cell profile is from 1 to 700 μm.

16. A plastic rod obtained according to the process of claim 8, comprising:
a hard compact profile; and
a foamed core rod;
wherein a cell diameter of the foamed rod is uniform.

17. A construction article comprising the plastic rod according to claim 16.

18. The process for producing a plastic rod according to claim 1, further comprising:
foaming the first plastic composition to obtain a foamed profile; and
foaming the second plastic composition to obtain a foamed core rod.

19. A medical implant produced by a method comprising the process according to claim 18.

20. The medical implant according to claim 19, wherein a cell structure of the profile is different from a cell structure of the core rod.
